(19) United States
(12) Patent Application Publication
(10) Pub. No.: US 2017/0330297 A1
(43) Pub. Date: Nov. 16, 2017

(54) DYNAMIC WEARABLE DEVICE BEHAVIOR BASED ON FAMILY HISTORY

(71) Applicant: KONINKLIJKE PHILIPS N.V., EINDHOVEN (NL)

(72) Inventors: John Cronin, Bonita Springs, FL (US); Roger Holmes, Son en Breugel (NL)

(21) Appl. No.: 15/528,390

(22) PCT Filed: Nov. 26, 2015
(86) PCT No.: PCT/EP2015/077710

§ 371 Date: May 19, 2017

Related U.S. Application Data
(60) Provisional application No. 62/087,727, filed on Dec. 4, 2014.

(30) Foreign Application Priority Data
Jun. 4, 2015 (EP) 15170681.9

(51) Int. Cl. G06Q 50/24; G06F 19/00; A61B 5/00

(52) U.S. Cl. CPC G06Q 50/24; G06F 19/345; A61B 5/6802; G06F 19/322; G06F 19/3431

(57) ABSTRACT

Various embodiments described herein relate to a server and related wearable device, method, and machine-readable storage medium including one or more of the following: receiving, at a rule installation server, family health data associated with at least one family member of a wearable device user; retrieving a candidate rule including installation criteria and an identification of a wearable device rule; evaluating the installation criteria using the family health data to determine that the candidate rule is to be installed; and based on determining that the candidate rule is to be installed, communicating the wearable device rule for installation on the wearable device.

### CROSS-REFERENCE TO RELATED APPLICATIONS

The present application claims the priority benefit of U.S. provisional application No. 62/087,727 filed Dec. 4, 2014 and entitled “Family History,” the disclosure of which is hereby incorporated by reference.

### TECHNICAL FIELD

Various embodiments described herein generally relate to wearable devices for collecting biometrics, motion, and other types of metrics about a wearing user. More specifically, but not exclusively, various embodiments relate to modifying the behavior of a wearable device based on the wearer's family history.

### BACKGROUND

Wearable technology includes mobile electronic devices that can be worn on the body or attached to or embedded in clothes and accessories of an individual. Processors and sensors associated with the wearable technology may be provided to gather, process, and display information to a user. Wearable technology may be utilized in a variety of areas including monitoring health data of a user and providing other types of data and statistics. Examples of wearable technology in the health arena include the FITBIT, NIKE+FUELBAND, and APPLE WATCH devices. Other wearable devices include the FREDERIQUE CONSTANT, MONDAINE, and ALPINA smartwatches.

Family histories are frequently used by doctors, nurses, and patients when predicting potential health risks of a patient. This is because in many cases, a person's risk factor for a condition may be increased when family members have suffered from the same condition or exhibit other warning signs. However, family health information is often difficult to reliably and consistently obtain.

### SUMMARY

Various embodiments of the present invention relate to methods for identifying health recommendations based on family history. Such methods may include receiving a family health history input regarding a user of a wearable device, detecting a health parameter measurement via a health parameter sensor of the wearable device, comparing the family health history input and the health parameter measurement to information stored in an action-rule database, and performing an action identified in the action-rule database when the family health history input and the health parameter match a rule associated with the identified action.

Further embodiments described herein include systems for identifying health recommendations based on family history. Such systems may include a wearable device. Such a wearable device may include memory that stores a family health history input associated with a user of a wearable device, a health parameter sensor that detects a health parameter measurement, and a processor that executes commands to compare the family health history input and the health parameter measurement to information stored in an action-rule database and to perform an action identified in the action-rule database when the family health history input and the health parameter match a rule associated with the identified action.

Additional embodiments described herein include non-transitory computer-readable storage media, having embodied thereon a program executable by a processor to perform a method for providing on-demand wireless services. Such a program may therefore include instructions for receiving a family health history input regarding a user of a wearable device, detecting a health parameter measurement via a health parameter sensor of the wearable device, comparing the family health history input and the health parameter measurement to information stored in an action-rule database, and performing an action identified in the action-rule database when the family health history input and the health parameter match a rule associated with the identified action.

Additional embodiments described herein include methods to facilitate the process of creating a personal family history tree, genogram or other means of visual representation or capturing of the content that contains all known relevant health parameters and facilitates automatic and or user initiated completing of the tree and manages levels of disclosure of information between family members and their respective electronic patient files. Such facilitation may be accomplished via user portal and controlled disclosure to family member settings.

Additional embodiments described herein include methods to facilitate the process of creating a personal family history tree, genogram or other means of visual representation or capturing of the content that contains all known relevant health parameters and facilitates automatic and or user initiated completing of the tree and manages levels of disclosure of information between family members and their respective electronic patient files. Such facilitation may be accomplished via privacy-sensitive capturing of data, guidance to conversations with family members about the subject, practical formats for capturing information, search & find tips for potential (online) sources of family health information, template localized approval forms for facilitation of elicitation of disclosure of family health information via electronic patient dossiers in line with local laws.

Additional embodiments described herein include methods to capture, store, and analyze available historic modifiable lifestyle behavior data of family members in order to recalculate non-modifiable behavior related family history risk assessments.

Additional embodiments described herein include methods to capture, store, and analyze available future modifiable lifestyle behavior data of family members in order to recalculate non-modifiable behavior related Family History risk assessments.

Additional embodiments described herein include methods to capture, store, and analyze available information about relevant confounding or contributing factors to modifiable lifestyle behavior data of family members and the extended ecosystem of the user. Examples include information about typical coping strategies in family, estimations of available absolute and perceived social support, information about family members historical absolute and perceived financial situations, information about personality related factors that may ameliorate or deteriorate the potentially detrimental health behaviors.

Various embodiments described herein relate to a method of configuring wearable device behavior based on family history, the method including: receiving, at a rule installation server, family health data associated with at least one family member of a wearable device user; retrieving a candidate rule including installation criteria and an identification of a wearable device rule; evaluating the installation criteria using the family health data to determine that the candidate rule is to be installed; and based on determining that the candidate rule is to be installed, communicating the wearable device rule for installation on the wearable device.

Various embodiments described herein relate to a rule installation server including: a memory storing a candidate rule including installation criteria and an identification of a wearable device rule; a network interface; and a processor configured to: receive family health data associated with at least one family member of a wearable device user, evaluate the installation criteria using the family health data to determine whether the candidate rule is to be installed; based on a determination that the candidate rule is to be installed, communicating the wearable device rule for installation on the wearable device.

Various embodiments described herein relate to a non-transitory machine-readable medium encoded with instructions for execution by a rule installation server, the non-transitory machine-readable medium including: instructions for receiving, at a rule installation server, family health data associated with at least one family member of a wearable device user; instructions for retrieving a candidate rule including installation criteria and an identification of a wearable device rule; instructions for evaluating the installation criteria using the family health data to determine whether the candidate rule is to be installed; and instructions for, based on a determination that the candidate rule is to be installed, communicating the wearable device rule for installation on the wearable device.

Various embodiments are described wherein communicating the wearable device rule for installation includes transmitting a message to the wearable device to effect installation of the wearable device rule.

Various embodiments are described wherein receiving the family health data includes receiving, from a user of the wearable device, an identification of one or more health conditions experienced by a family member.

Various embodiments are described wherein receiving the family health data includes accessing an electronic health record of at least one family member.

Various embodiments additionally include receiving, from the at least one family member, an identification of relationship to the wearable device user; and based on receiving the identification of relationship to the wearable device user, modifying a user record of the wearable device record to reflect a permission to access health data of the at least one family member, wherein receiving the health data includes retrieving the health data of the at least one family member based on the permission.

Various embodiments additionally include receiving, from the wearable device user, an identification of relationship to the at least one family member; transmitting, to the at least one family member, a request to grant permission to access health data of the at least one family member; receiving, from the at least one family member, an approval to access health data of the at least one family member; based on receiving the approval, modifying a user record of the wearable device record to reflect a permission to access health data of the at least one family member, wherein receiving the health data includes retrieving the health data of the at least one family member based on the permission.

Various embodiments are described wherein evaluating the installation criteria using the family health data includes evaluating at least one modifiable risk factor of the at least one relative.

### BRIEF DESCRIPTION OF THE DRAWINGS

In order to better understand various example embodiments, reference is made to the accompanying drawings, wherein:

FIG. 1 illustrates an example of a computer networked environment where a wearable device, an optional user device, a third party network, a wearable device vendor network, and a doctor network may communicate over a packet data network;

FIG. 2 illustrates an example of a family history profile where one or more family health risks may be selected and passed to a variety of wearable device types that could use this information;

FIG. 3 is a flow chart illustrating an example of a method performed by physician or other server for selecting rules for installation;

FIG. 4A illustrates an example of family history software interacting with a doctor server;

FIG. 4B illustrates an example of an action rule database snapshot that includes a series of rules, actions types associated with the rules, and specific actions associated with the rules;

FIG. 5 illustrates examples of where family history data may be sent after entered into the family history software by a user;

FIG. 6 illustrates an example of a mobile device architecture that may be utilized to implement the various features and processes described herein;

FIG. 7 illustrates an example of a candidate rule database that may be used by a server for installing rules;

FIG. 8 illustrates an example of a method of correlating family history and data collected by a wearable device to a health risk;

FIG. 9 is a flowchart illustrating an example of a method for requesting and establishing sharing of health information between a patient and a family member;

FIG. 10 is a flow chart illustrating an example of a method for confirming or denying requests for access to health information;

FIG. 11 is a flowchart illustrating an example of a method for establishing sharing of health information after grant of permission;

FIG. 12 is a flowchart illustrating an example of a method for identifying rules for installation using family history information;

FIG. 13 illustrates an example of a candidate rule database;

FIG. 14 illustrates an example of a family history criteria formula; and

FIG. 15 illustrates an example of hardware for implementing a rule installation server.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or,” as used herein, refers to a non-exclusive or (i.e., or), unless otherwise indicated (e.g., “or else” or “or in the alternative”). Additionally, the various embodiments described herein are not necessarily mutually exclusive and may be combined to produce additional embodiments that incorporate the principles described herein.

Various embodiments described herein achieve various functionality through the execution of instructions by a processor. It will be understood that, while various examples are described in the context of instructions actively performing steps or other actions, any such actions will actually be performed by the processor that executes such instructions.

While wearable electronic devices include capabilities of monitoring indications of health (e.g., blood pressure, body temperature, blood sugar level, motion) via sensors/accelerometers, these wearable devices are not aware of family history and cannot cross reference family health history with measurements made by the wearable devices. Currently, a user (or a doctor of the user) would need to manually cross-reference family health history information in order to see if the user's family history is affecting the user's health as measured by the sensors of the wearable device. This is a cumbersome and slow process, and by the time the information is procured, it might no longer be relevant or useful to the user or to the doctor.

According to the foregoing, it would be beneficial to provide improved systems and methods for cross-referencing family history with measurements made by a wearable device to help improve the health of a user of the wearable device.

Various embodiments described herein generally relate to a system and method for cross-referencing data measured by a wearable device with family history data. Data sensed by sensors at the wearable device is reviewed for health risks that correspond to known medical conditions of a family member. In certain instances, a recommendation may be made to the user that when followed will improved the health of the user.

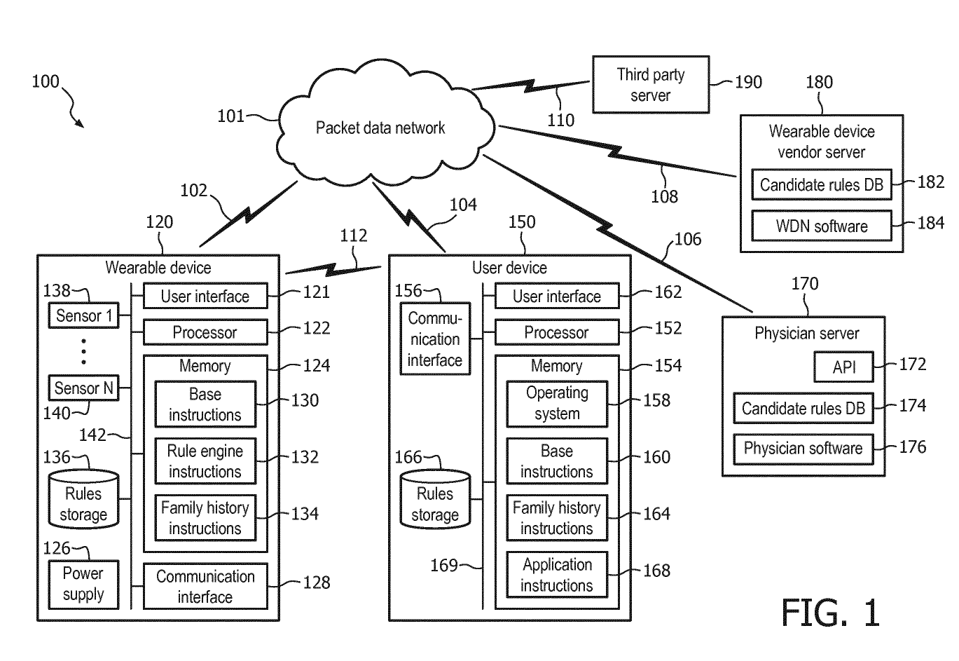

FIG. 1 illustrates a computer networked environment 100 where a wearable device 120 provided by a vendor, a user device 150, a third party server 190, a wearable device vendor server 180, and a physician server 170 may communicate over a packet data network 100. The network environment 100 includes communication pathways 102, 104, 106, 108, 110, and 112, where communication pathways 102, 104, 106, 108, and 110 may traverse the packet data network 101. Communication pathway 112 may be a direct communication path that may be used when the wearable device 120 communicates directly with the user device 150. Each of these communication pathways may be a wireless or a wired communication path known in the art including, yet not limited to Universal Serial Bus (“USB”), a FireWire connection, a Lightning connection, a Thunderbolt connection, Bluetooth, Bluetooth Low Energy, Bluetooth Smart, Wi-Fi, cellular (2G, 3G, 4G, LTE, Edge), or Ethernet communication pathways.

The packet data network 101 may include, for example, a carrier network, a local area network (LAN), or a wide area network (WAN) such as the Internet. As such, the packet data network 101 may provide access to various servers, including the illustrated servers 170, 180, 190 and other servers not illustrated. It will be apparent that various servers, such as one or more of the servers 170, 180, 190, may be provisioned as virtual machines within a cloud computing environment. Various references to “the cloud” used herein will be understood to refer to various services or resources provided to external users from within such a cloud computing environment.

The wearable device 120 may include one or more sensors 1-N (illustrated as sensor 1 138 and sensor N 140), a processor 122, a memory 124, a power supply 126, a communication interface 128, a user interface 121, and a rules storage 136 in communication via a system bus 142. It will be apparent that various alternative sets of components and arrangements thereof may be utilized. For example, additional buses, such as a peripheral bus, may be used or one or more of the sensors 138, 140 may be implemented as external devices that separately attach to the wearer's body and communicate with the wearable device via a wired or wireless connection such as, for example, via the communication interface 128. In various embodiments, the communication interface 128 may be a USB port, a FireWire, a Lightning, a Thunderbolt, a Wi-Fi, a 3G/4G/LTE cellular, a Bluetooth, a Bluetooth low energy, a, Bluetooth Smart, a near field communication, or a radio wave interface.

The one or more sensors 138, 140 may include any type of sensor that is known in the art. Generally, the sensors 138, 140 may be used, for example, to detect and obtain sensor data (e.g., biometrics) about the user (e.g., heart rate, blood pressure) or obtain sensor data regarding the surrounding environment (e.g., temperature, humidity). Sensors may also be used for other purposes such as a step counter (e.g., pedometer). The sensors 138,140 may be mounted on the wearable device 120 or may be external devices that are separately wearable by the user and communicate with the wearable device 120 wirelessly or via a wired connection.

The user interface 121 may include various hardware for interacting with a user, such as the wearer of the wearable device. As such, the user interface 121 may include, for example, a video display or other display device, a touchscreen input which may be positions over the display device, one or more buttons, a keypad, a speaker, a camera, or a haptic feedback engine.

The power supply 126 may be used to provide power used by the wearable device 110 for maintaining operation of the overall device. In various embodiments, the power supply 126 may include a battery, one or more capacitors, a powered USB interface, or a power cord and plug. In some embodiments, the power supply may be chargeable using an external power source (e.g., battery charger).

The rules storage 136 may be a storage device such as read-only memory (ROM), random-access memory (RAM), magnetic disk storage media, optical storage media, flash-memory devices, or similar storage media. In various embodiments the rules storage 136 may store a rules database (examples of which will be described below) which provides rules for affecting the behavior of the wearable device 120. As used herein, the rules storage 136 may be referred to as a rules database 136 when referring to the database stored in the storage device 136.

As shown, the memory 124 stores base instructions 130, rule engine instructions 132, and family history instructions 134 for execution by the processor 122, though it will be apparent that various additional sets of instructions may also be stored in the memory 124. For example the memory 124 may store an operating system, weather instructions, and graphical user interface (GUI) instructions. It will be understood that these instructions may be alternatively or additionally stored in a non-volatile storage device such as the rules storage 136 or another storage device (not shown). For example, the instructions may be stored in a flash memory or an electronic read only memory (ROM) until they are to be executed by the processor, at which point they are copied to the memory 124. As used herein, the term storage will be understood to refer to non-volatile memories.

As will be understood, devices referred to herein as “storage” or “memory” may both be considered to be “non-transitory machine-readable media.” As used herein, the term “non-transitory” will be understood to exclude transitory signals but to include all forms of information storage, including both volatile and non-volatile memories.

The base instructions 130 may be used by the processor 122 to conduct the various processes and calculations for the wearable device 110. The specific implementation of the base instructions 130 will be highly dependent on the overall goal or purpose of the wearable device 110; for example, a wearable device for tracking a heart rate will include different base instructions 130 from a wearable device for tracking steps taken. For example, the base instructions 130 may be used to calculate one or more parameters based on measured sensor data obtained from the plurality of sensors 138, 140. For example, in some embodiments wherein the sensors 138, 140 include a step counter, and the base instructions 130 may be used to take the number of steps taken by the user and calculate possible parameters such as distance traveled by the user or number of calories burned by the user.

The rule engine instructions 132 may be used by the processor 122 to evaluate and apply rules stored in the rules storage 136. In various embodiments, the rule engine instructions may be invoked periodically, upon creation of new sensor data by the sensors 138, 140, upon creation of new parameter data through operation of the base instructions 130, upon receiving user input, upon receiving a prompt via the communication interface 128, or in response to other stimulus. In some embodiments where rules include applicability criteria and resultant actions, the rule engine instructions 132 may iterate through available rules in the rules storage and compare applicability criteria to the current context (e.g., recent measured sensor data or parameters or, in some embodiments, family history data) to determine whether each rule is applicable. Upon identifying an applicable rule, the rule engine instructions 132 may go on to effect performance of the resultant action(s) defined by the rule. For example, a rule may indicate that a textual, graphical, video, audio, or tactile message be output to the user; a message including predefined or measured data be transmitted to another device such as, for example, the user device 150 or one of the servers 170, 180, 190; additional sensor measurements be taken; or additional parameters be calculated.

The family history instructions 134 may be used by the processor 122 to enable user entry of data related to the wearing user's family history. For example, the family history instructions 134 may enable a user to enter, via the user interface 121, one or more indications of health conditions experienced by a family member. In various embodiments, the family history instructions 134 may alternatively or additionally enable a user to enter, via the user interface 121, an identification of one or more family members for which health data is to be retrieved or requested. For example, upon identification of a family member, the family history instructions may transmit a permissions request to the family member or a representative thereof (e.g., one of the servers 170, 180, 190) for access to, e.g., electronic health records or wearable device data. In some embodiments, the family history instructions 134 may alternatively or additionally enable a user to grant or deny access of requesting family members to the user's own health data.

The processor 122 may be virtually any device capable of performing the functions described herein including the functions described above in connection with the base instructions 130 and family history instructions 134. For example, the processor 122 may include one or more microprocessors, one or more field-programmable gate arrays (FPGA), or one or more application-specific integrated circuits (ASIC). In some embodiments, the processor may not utilize stored instructions to perform some or all of the functions described herein; for example, an ASIC may be hardwired to perform one or more of the functions describe above with reference to the base instructions 130 and family history instructions 134. In some such embodiments, the base instructions 130 and family history instructions 134 may be omitted because they are already embodied in the processor 122 without the need for stored instructions.

The wearable device 120 may connect to packet data network 101, and ultimately to the other devices depicted in FIG. 1, through connection 102. In some embodiments, the wearable device 120 may also connect directly to a user device 150, such as a mobile phone, tablet, or computer, through a connection 112. These connections may be performed through communication interface 128. In some embodiments, the elements of wearable device 120 are all connected to one another through a single bus 142, while in other embodiments, the wearable device include two or more buses arranged to interconnect the component. It should be understood that the components of wearable device 120 as illustrated in FIG. 1 and described above are illustrative rather than limiting. A wearable device 120 need not include all of these components and/or may include additional components not listed herein.

Some embodiments may include a user device 150 to supplement the operation of the wearable device 120. User device 150 may include, for example, a smartphone, a tablet, a laptop computer, a desktop computer, a gaming console, a smart television, a home entertainment system, a second wearable device, or another computing device that may provide additional computing functionalities to the wearable device 120. The user device 150 may include a wired and/or wireless communication interface 156 (e.g, a USB port module, a FireWire port module, a Lightning port module, a Thunderbolt port module, a Wi-Fi connection module, a 3G/4G/LTE cellular connection module, a Bluetooth connection module, a Bluetooth low energy connection module, a, Bluetooth Smart connection module, a near field communication module, a radio wave communications module), a rules storage 166, a user interface 162, a processor 152, and a memory 154. In some embodiments, rather than maintaining a local rules storage 166 at the user device 150, a rules database may be stored within a local network, or may be accessed by other devices within the local network. The user device 150 may connect to the packet data network 101, and ultimately to the other devices 170, 180, 170 depicted in FIG. 1, through connection 104. In some embodiments, the user device 150 may also connect directly to the wearable device 120 through a wired or wireless connection 112. These connections may be performed through communication interface 156. In some embodiments, the elements of user device 150 may communicate with each other using a single communication bus 169, while in other embodiments, the user device may have more of a divided architecture. It will be understood that the components of user device 150 as illustrated in FIG. 1 and described above are illustrative rather than limiting. A user device 150 need not include all of these components and/or may include additional components not listed herein.

In various embodiments, the communication interface 156, user interface 162, processor 152, memory 154, and rules storage 166 may include similar physical devices to those described above with respect to the communication interface 128, user interface 121, processor 122, memory 124, and rules storage 136 described above. As used herein, the rules storage 166 may be referred to as a rules database 166 when referring to the database stored in the storage device 166. As shown, the memory 154 may store various instructions for execution by the processor such as, for example, an operating system 158, base instructions 160, family history instructions 164. The operating system 158 may coordinate the various basic functions of the user device 150. For example, where the user device 120 is a mobile phone or tablet, the operating system 158 may be the APPLE IOS or GOOGLE ANDROID operating system.

The base instructions 160 may include various instructions for causing the processor to perform or supplement the base operations of the wearable device 138, 140. For example, in some embodiments, the wearable device 120 may not calculate any parameters; instead, the base instructions 130 of the wearable device 120 may simply gather sensor data and transmit the data to the user device 150. The base instructions 160 of the user device may then use the data to calculate one or more parameters or locate applicable rules in the rules storage 166. As another example, in some embodiments, the base instructions 130 of the wearable device 120 may calculate “instantaneous parameters” such as, for example, the number of steps taken in the current reporting cycle while the base instructions 160 of the user device 150 may use these instantaneous parameters to calculate aggregated parameters such as, for example, steps taken today or average steps taken per day over the last week.

The family history instructions 164 may be similar to the family history instructions 134 described above with respect to the wearable device. For example, the family history instructions 134 may enable a user to enter, via the user interface 121, one or more indications of health conditions experienced by a family member. In various embodiments, the family history instructions 134 may alternatively or additionally enable a user to enter, via the user interface 121, an identification of one or more family members for which health data is to be retrieved or requested. For example, upon identification of a family member, the family history instructions may transmit a permissions request to the family member or a representative thereof (e.g., one of the servers 170, 180, 190) for access to, e.g., electronic health records or wearable device data. In some embodiments, the family history instructions 134 may alternatively or additionally enable a user to grant or deny access of requesting family members to the user's own health data.

The application instructions 168 may be used by the processor to present to a user, via the user interface, a user application associated with the wearable device. For example, the application instructions 168 may present histograms of reported sensor data or calculated parameters. Additionally or alternatively, the application instructions 168 may present a graphical user interface for entering family history data that, upon entry, invokes the family history instructions 164. As such, in some embodiments, the application instructions 168 may encompass the base instructions 160 or family history instructions 160.

The wearable device vendor server 180 may be operated by a vendor of the wearable device 120 and may include various components such as a candidate rule database 182 and a wearable device network (WDN) software 184. These may each be hosted on one or more servers or networked computing devices or virtual machines. In some embodiments, some of these elements may be missing and/or additional elements may be part of the wearable device vendor server 180. The wearable device vendor network 180 may connect to the network 101, and ultimately to the other devices depicted in FIG. 1, through connection 108.

The physician server 170 may be operated by a physician of the wearable device 120 user and may include a candidate rules database 174, physician software 176, and an application program interface (API) 172. These may each be hosted on one or more servers or networked computing devices or virtual machines. In some embodiments, some of these elements may be missing or additional elements may be part of the physician server 170. The physician server 170 may connect to the network 101, and ultimately to the other devices depicted in FIG. 1, through connection 106,

In some embodiments, a third party server 190 may also be present, The third party server 190 may connect to the network 101, and ultimately to the other devices depicted in FIG. 1, through connection 110. In some embodiments, the third party server may be a weather server, a health weather server (e.g., which may provide information about allergens in the air, toxins in the air/water, or other environmental health dangers), a health server, a gymnasium server, a food/dietary server, a fitness server, an emergency services server, a caregiver server, a patient support server, an ancestry data server, or another type of server.

When a user device 150 is used in various embodiments, the user device 150 may be tethered to the wearable device 120 with a wired or wireless connection 112 (e.g., a network connection, a Bluetooth connection, a USB connection). The user device 150 may in some embodiments be used as a proxy for the wearable device 120. When this occurs, the user device 150 may receive information from the wearable device 120 through connection 112, and the user mobile device 150 may communicate this information over the network 101 to a receiver of this information (e.g., the physician server 170, wearable device vendor server 180, or a 3rd party server 190 such as a weather server or a health weather server). Alternatively, the wearable device 120 may send an information request to the user mobile device 150, which may then connect to the network 101, retrieve the requested information from a data source (e.g., the physician server 170, wearable device vendor server 180, or 3rd party server 190 such as a weather server or a health weather server), and transmit the requested information back to the wearable device 120 using the connection 112. The user device 150 may also display recommendations received from an network 101 data source such as a third party server 190 (e.g., health weather server) on a display, as well as communicate information (e.g., weather data) received to the wearable device 120. The advantage of a user mobile device 150 acting as a proxy may derive from the situation where a user mobile device 150 may have greater processing and communication capabilities than the wearable device 120. For example, a wearable device 120 may, in some embodiments, not be capable of communicating over a cellular network, where a user mobile device 150 may be able to communicate over both a cellular and a Bluetooth network. For example, sensor data from sensors 1-N (138-140) may be used to sense motion or activity of a user of a wearable device and that motion data may be used to calculate a number of steps stepped, or a number of calories burned during the sensed motion.

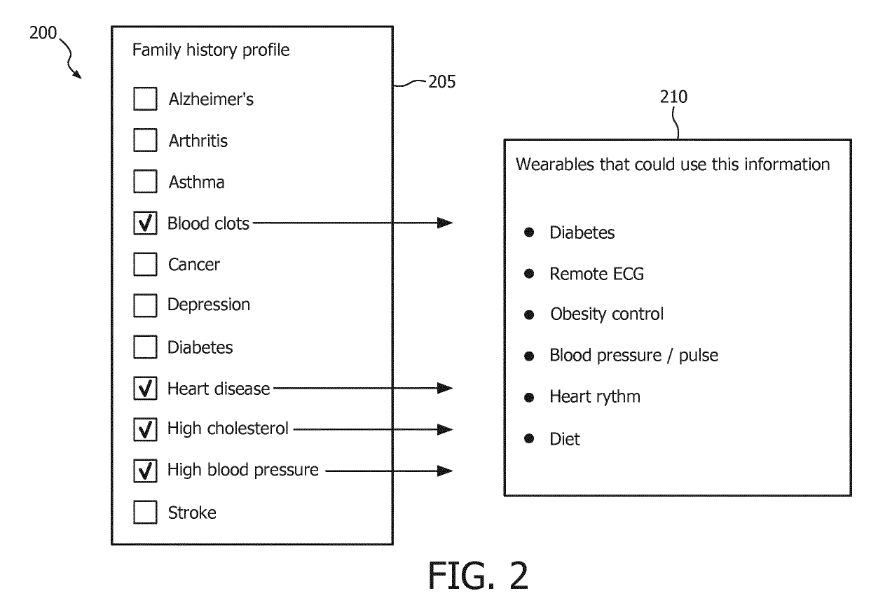

FIG. 2 illustrates an information flow 200 where one or more family health risks may be selected in a family history profile 205 and passed to a variety of wearable device types 210 that could use this information. The family health history profile 205 includes a plurality of health risk selection boxes that identify health risks that have affected a family member (e.g., Alzheimer's, arthritis, asthma, blood clots, cancer, depression, diabetes, heart disease, high cholesterol, high blood pressure, stroke). While the example family history profile 205 interface of FIG. 2 illustrates many health risk selection boxes that may be selected for the family history profile 205, FIG. 2 depicts an example interface in which a user has selected blood clots, heart disease, high cholesterol, and high blood pressure as family history health conditions. It should be understood that this list is illustrative rather than limiting, and a family history profile could list numerous addition conditions, diseases, or birth defect. In some embodiments, it may also list medication histories, average death ages, infant mortality rates, mutations, and other family health history traits that could be helpful to a medical professional.

Numerous types of wearable devices 210 could use information from such a family history profile 205. Some example wearable devices that could use this family history profile information 200 may include wearable devices built for diabetes care, remote electroencephalogram (EEG) measurement, obesity control, blood pressure/pulse measurement, heart rhythm measurement, and diet control. Other types of wearable devices could also use such family history profile 200 information.

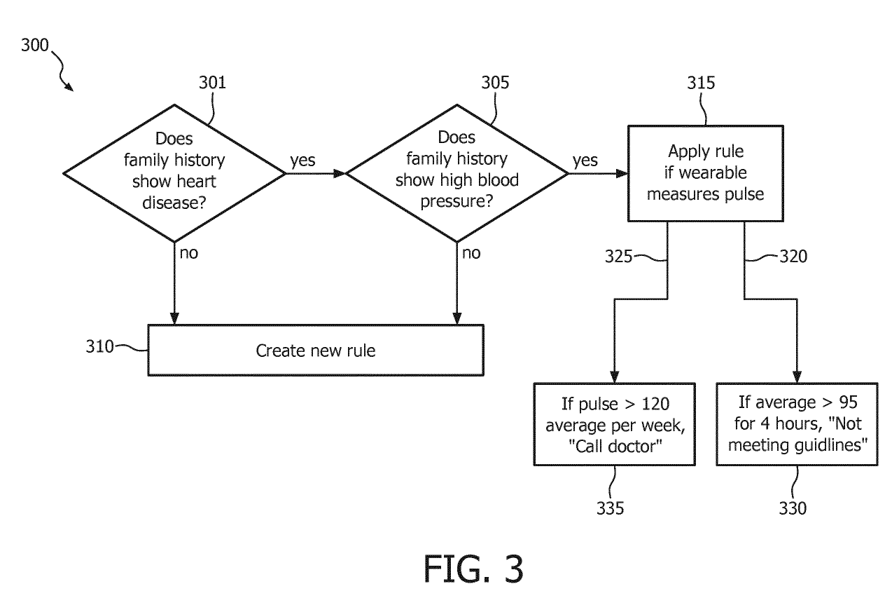

FIG. 3 illustrates a flow chart 300 of an example operation of the physician software 176. A first determination step in the example embodiment of FIG. 3 determines whether family history shows heart disease in step 301. If the family history does show heart disease, the flow chart moves to a second determination step, which is to determine whether the family history shows high blood pressure in step 305.

If the family history does show high blood pressure, a rule-action combination may be generated and applied based on this history. For example, if a user has a family history of high blood pressure, a rule that might otherwise provide a “high blood pressure” warning action at a specific threshold blood pressure could be adjusted to provide that “high blood pressure” warning action at a lower threshold blood pressure. Regardless, the doctor's software next receives a sensor measurement from the wearable device 120, which according to the example embodiment of FIG. 3 can be a measurement of a pulse by the wearable device 120 in step 315. This doctor's software can then look through a rule database (such as candidate rule database 174, or rule database 166, or rule database 136, or candidate rule database 182, or another rule database) to determine a rule to check the measured pulse against. Depending on the measured pulse, and depending on the set of rules from the rule database (174 or otherwise), the doctor's software program flow can proceed along a first path (e.g., path 320) to an example first rule 330 or along a second path (e.g., path 325) to an example second rule 335. According to the first rule 315, when a user's pulse rate averages at greater than 95 beats per minute for 4 hours, a determination is made that the individual's heart rate is not meeting guidelines in step 330. The rule database (174 or otherwise) can then recommend an action to take, such as sending a “not meeting guidelines” message to the user of the wearable device 120. When the user's average heart rate averages at greater than 120 beats per minute over a week, a determination is made that the user's doctor should be contacted (e.g., via a phone call, text message, alert on a physician portal, alert to a central practitioner post, a trigger sent to an immediate care network, etc.). The rule database (174 or otherwise) can then recommend an action to take, such as sending a “call doctor” message to the user of the wearable device 120, or automatically triggering a phone call or e-mail to the doctor's office.

If the first determination step instead determines that the family history does not show heart disease in step 301, the example doctor's software 176 instead creates a new rule in step 310. Similarly, if the second determination step 305 instead determines that the family history does not show high blood pressure in step 305, the example doctor's software 176 also creates a new rule in step 310. In operation, a doctor interacting with the software may create new rules in the doctors software manually (e.g., set a wider health range for a user who the doctor knows exercises heavily) or automatically (e.g., automatically adjust a health range based on prior activity and health, or based on family history). In some embodiments, a doctor may manually create a rule (as in step 310) at any time regardless of family history or measurements.

It should be understood that FIG. 3 shows an example embodiment, and that the invention is not limited to family history profiles of heart disease and high blood pressure, nor wearable device measurements of a user's pulse. For example, the wearable device could include sensors measuring hydration, calories, blood pressure, blood sugar or glucose, insulin, body temperature (i.e., thermometer), heart rate, weight, sleep, number of steps (i.e., pedometer), velocity or acceleration (i.e., accelerometer), vitamin levels, respiratory rate, heart sound (i.e., microphone), breathing sound (i.e., microphone), movement speed, skin moisture, sweat detection, sweat composition, nerve firings (i.e., electromagnetic sensor), or similar health measurements. Likewise, the family history profiles may track, for example, family incidents of Alzheimer's, arthritis, asthma, blood clots, cancer, depression, diabetes, heart disease, high cholesterol, high blood pressure, or strokes in the family of the user of the wearable device.

While the flow diagram in FIG. 3 shows a particular order of operations performed by certain embodiments described herein, it should be understood that such order is an example (e.g., alternative embodiments can perform the operations in a different order, combine certain operations, overlap certain operations, etc.).

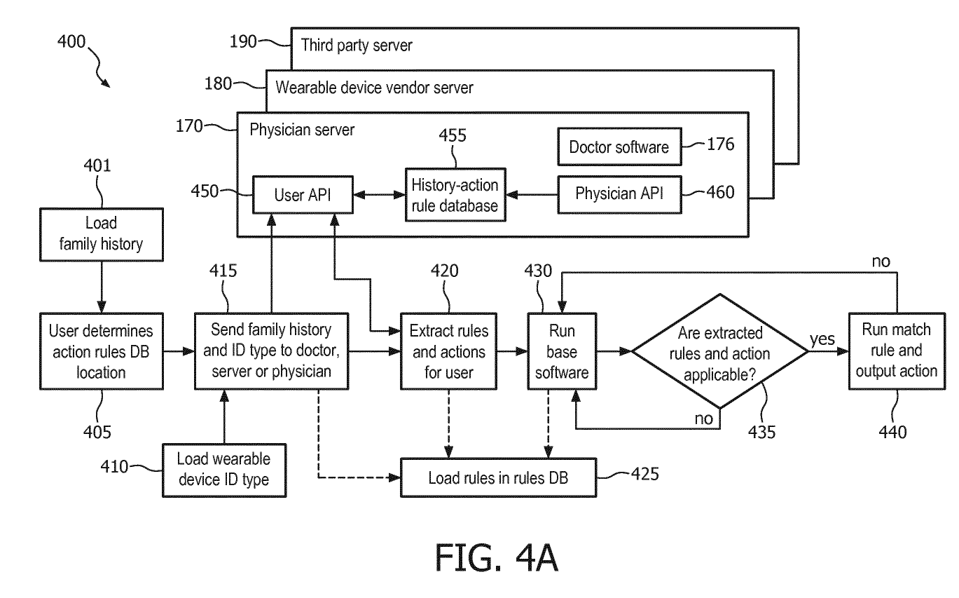

FIG. 4A illustrates an example implementation 400 of family history instructions 134 interacting with a physician server 170. In step 401 of the family history instructions 134, a family history profile (e.g., family history profile 201) may be loaded into a user interface by a user. In step 405, action rules may be determined or entered into a database by the user. In step 415, the family history information may be sent to a physician/doctor/caregiver, or to a doctor server 170. Step 415 may send this family history into a rules database 136, or step 425 may load rules in a rules database 166 or the history-action rule database 455 (one embodiment of the action rules database 174) through a user API 450, a subset of the doctor network API 172. Step 415 may also receive data identifying a wearable device (by its “type” or sensor capabilities or by a device identifier) and/or data including various types of identified health data from the wearable device (e.g., step 410). Afterwards, the family history instructions 134 may extract rules and actions associated with the user (e.g., step 420) of the user into rules database 136 or rules database 166 (e.g., step 425), or into the history-action rule database 174, which may be done through the user API 450 (e.g., step 420). In some embodiments, the history-action rule database 174 may also be modified by a physician API 460, a second subset of the API 172. In some embodiments, the history-action rule database 174 may be accessed by the doctor's software 176.

After the rules extraction step 420, the base instructions 130 are run (e.g., step 430), and program flow moves to a first determination step. The first determination step determines whether rules have been extracted and whether those rules are applicable to a recommended action (e.g., step 435). When the first determination step determines that a recommended action corresponds to the extracted rules, the next step 440 of the flow chart matches the rule to the recommended action. In various embodiments, steps 435, 440 may correspond to the rules engine instructions 132 of FIG. 1. Program flow then flows back to running the base software 130 in step 430. Each time the program flow flows back to running the base software, the program may load rules into the rules database 136 or rules database 166 in step 425.

In an alternate embodiment, FIG. 4A may illustrate example operations of family history software 164 of the user mobile device 150 rather than family history software 134 of the wearable device 120, In such an embodiment, the “base software” of step 430 refers to base software 160 of the user mobile device 150 rather than base software 130 of the wearable device 120.

A series of dashed lines in the figure indicates that new rules may be loaded into a rules database in step 425. Optionally the step were new rules are accessed may be accessed from the send family history step 415, from the extract rules and action step 420, or from the run base software step 430. The rules database of step 425 may refer to rules database 136, rules database 166, or history-action rules database 455, candidate rules database 174, candidate rules database 184.

The API in the physician server 170 may communicate with a history action rule database 455 in the physician server 170 (or network to which it belongs) which in turn may receive information from a physician doctor API 460. Doctor software 176 as discussed in respect to FIG. 3 is also included in the physician server 170. The figure also depicts a wearable device server 180 and a third party server 190 that may receive user information provided from the family history software 134 or 164 and interact with the wearable device or user device in a manner similar to that described with respect to the physician server 170.

In some embodiments, an additional step (not shown) may be added between determining applicability of a rule in step 435 and performing the action associated with that rule in step 440. This additional step would check to ensure that the conclusion that the rule is applicable in step 435 would need to be met by multiple variations of the family history software 134 or 164 before the action is performed. For instance, the wearable device 120 may execute its family history instructions 134 or rule engine instructions 136 using its local rules database 136, and come to the conclusion that a rule is met, while a user mobile device 150 executes its family history instructions 166 using its local rules database 166 and comes to the conclusion that no rule is met, ultimately meaning that the action-rules databases are not aligned, or the family history profiles are not synchronized. According to one embodiment, this could mean that the action is ultimately not performed in step 440. In another embodiment, it could instead trigger a synchronization among action-rule databases. In some embodiments, a version of the family history software may also be executed by one of the networks (e.g., the physician server 170, the wearable device vendor server 180, or a third-party server 190). In one embodiment, then, a wearable device must come to the same action conclusion in step 435 as the physician server does in step 435 in order for the action to be performed in step 440.

While the flow diagram in FIG. 4A shows a particular order of operations performed by certain embodiments described herein, it should be understood that such order is an example (e.g., alternative embodiments can perform the operations in a different order, combine certain operations, overlap certain operations, etc.).

FIG. 4B illustrates an example action rule database snapshot 480 that includes a series of rules 485, actions types associated with the rules 490, and specific actions associated with the rules 495. In the example action rule database snapshot 480 of FIG. 4B, the action type 490 associated with all of these rules is to send a message a user of a wearable device 120 when a rule is met, indicated by the label “MSG” under the action type column 490. A first rule 485 triggers an action 490 when the calories consumed by the user are less than (<) 1800 calories per day. The action 495 matched to this first rule is to send a message that the user is “not meeting guidelines.” Other rules illustrated in the example action rule database snapshot 480 of FIG. 4B. Action rule database snapshot 480 includes a second rule indicating that when the user's amount of calories consumed is less than (<) 1800 calories per day in a row for 5 days, the action is performed of sending a message “call doctor's office.” Action rule database snapshot 480 includes a third rule indicating that when an average pulse rate is greater than (>) 95 beats per minute for 4 hours, the action is performed of sending the message “not meeting guidelines.” Action rule database snapshot 480 includes a fourth rule indicating that when an average pulse rate is over (>) 110 beats per minute over a week, the action is performed of sending the message “call doctor's office.” These entries should be understood to be illustrative rather than limiting.

While the action type 490 of all of the actions illustrated in FIG. 4B is to send a message, other actions are possible. For example, a rule could exist that triggers a nearby medical device, such as a kiosk blood pressure monitor or medical imaging machine, to provide a medical-grade sensor reading. The wearable device 120 could then interface with the medical device to obtain the reading through a downloading or synchronization process, or could display a message asking the user to input the reading from the medical device manually.

Alternatively, another action listed in action type 490 could be to trigger a phone call or message to another device. For instance, the second and fourth rules of FIG. 4B could, instead of triggering a user message telling the user to call a doctor's office, instead trigger and automatic phone call to the doctor's office, or send an automatic e-mail or text message to the doctor's office. Alternatively, instead of calling or messaging the doctor's office, the rule could trigger calling or messaging an emergency contact of the user, such as a family member, caregiver, or emergency services professional.

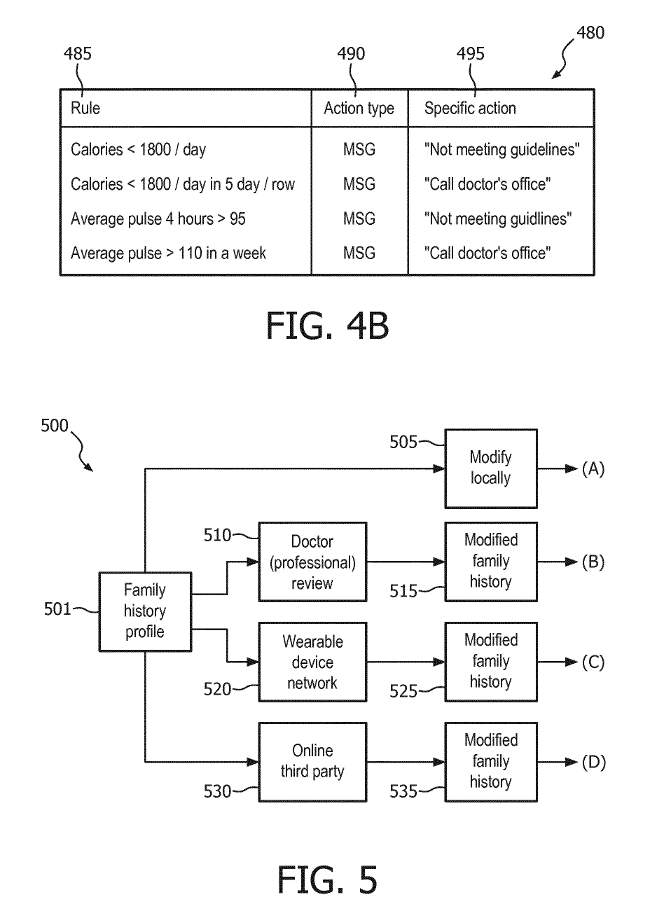

FIG. 5 illustrates optional locations 500 identifying examples of where family history data may be sent after entered into the family history software (134 or 164) by a user. A first box in the figure is where a user of the family history software (134 or 164) enters their family history profile 501. Family history profile 200 of FIG. 2 is an illustration of an example family history profile 501. In certain embodiments, the family history profile 501 may be entered through a GUI 162 displayed on a user device 150 or a GUI 121 a wearable device 120. The family history profile 501 may then be sent to a doctor's computer (e.g., through the doctor network 170), for a professional review 510. The family history profile 501 may alternately be sent to a wearable device vendor network 180 (block 520). The family history profile 501 may alternately be sent to an online third party network 190 (block 530). The family history profile 501 may alternately be stored locally on an electronic device (block 505). Once sent to each respective location identified by the user, the user family history may be modified at the doctor's computer or doctor network 170 (block 515), at the wearable device network (block 525), at the online third party network (block 535), or locally (block 505).

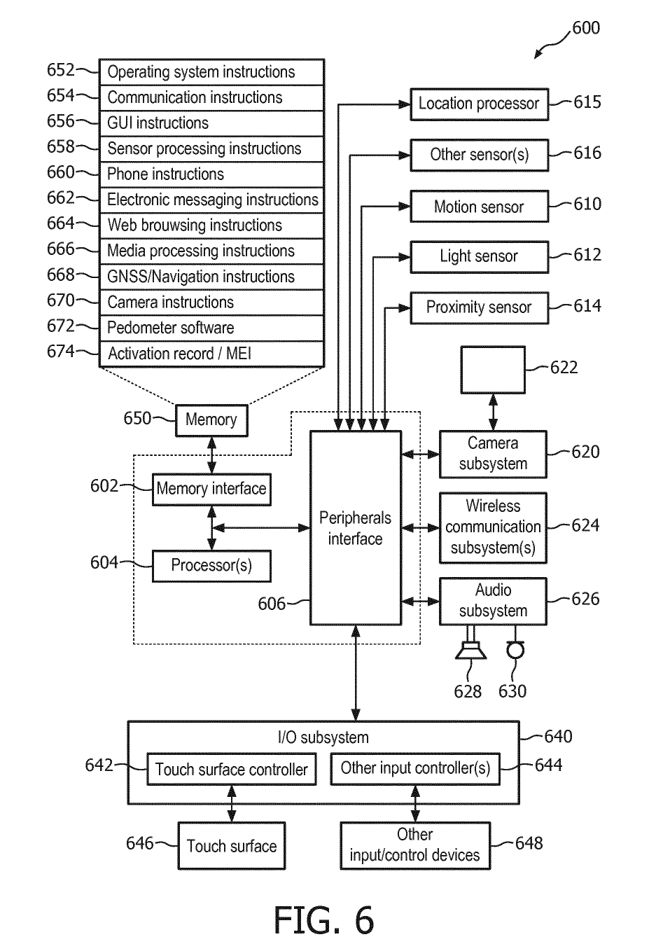

FIG. 6 illustrates a mobile device architecture that may be utilized to implement the various features and processes described herein. Architecture 600 can be implemented in any number of portable devices including but not limited to smart wearable devices such as wearable device 120 or a user device such as user device 150. Architecture 600 as illustrated in FIG. 6 includes memory interface 602, processors 604, and peripheral interface 606. Memory interface 602, processors 604 and peripherals interface 606 can be separate components or can be integrated as a part of one or more integrated circuits. The various components can be coupled by one or more communication buses or signal lines.

Processors 604 as illustrated in FIG. 6 are meant to be inclusive of data processors, image processors, central processing unit, or any variety of multi-core processing devices. Any variety of sensors, external devices, and external subsystems can be coupled to peripherals interface 606 to facilitate any number of functionalities within the architecture 600 of the exemplar mobile device. For example, motion sensor 610, light sensor 612, and proximity sensor 614 can be coupled to peripherals interface 606 to facilitate orientation, lighting, and proximity functions of the mobile device. For example, light sensor 612 could be utilized to facilitate adjusting the brightness of touch surface 646. Motion sensor 610, which could be exemplified in the context of an accelerometer or gyroscope, could be utilized to detect movement and orientation of the mobile device. Display objects or media could then be presented according to a detected orientation (e.g., portrait or landscape).

Other sensors could be coupled to peripherals interface 606, such as a temperature sensor, a biometric sensor, or other sensing device to facilitate corresponding functionalities. Location processor 615 (e.g., a global positioning transceiver) can be coupled to peripherals interface 606 to allow for generation of geo-location data thereby facilitating geo-positioning. An electronic magnetometer 616 such as an integrated circuit chip could in turn be connected to peripherals interface 606 to provide data related to the direction of true magnetic North whereby the mobile device could enjoy compass or directional functionality. Camera subsystem 620 and an optical sensor 622 such as a charged coupled device (CCD) or a complementary metal-oxide semiconductor (CMOS) optical sensor can facilitate camera functions such as recording photographs and video clips.

Communication functionality can be facilitated through one or more communication subsystems 624, which may include one or more wireless communication subsystems. Wireless communication subsystems 624 can include 802.5 or Bluetooth transceivers as well as optical transceivers such as infrared. Wired communication system can include a port device such as a Universal Serial Bus (USB) port or some other wired port connection that can be used to establish a wired coupling to other computing devices such as network access devices, personal computers, printers, displays, or other processing devices capable of receiving or transmitting data. The specific design and implementation of communication subsystem 624 may depend on the communication network or medium over which the device is intended to operate. For example, a device may include wireless communication subsystem designed to operate over a global system for mobile communications (GSM) network, a GPRS network, an enhanced data GSM environment (EDGE) network, 802.5 communication networks, code division multiple access (CDMA) networks, or Bluetooth networks. Communication subsystem 624 may include hosting protocols such that the device may be configured as a base station for other wireless devices. Communication subsystems can also allow the device to synchronize with a host device using one or more protocols such as TCP/IP, HTTP, or UDP.

Audio subsystem 626 can be coupled to a speaker 628 and one or more microphones 630 to facilitate voice-enabled functions. These functions might include voice recognition, voice replication, or digital recording. Audio subsystem 626 in conjunction may also encompass traditional telephony functions.

I/O subsystem 640 may include touch controller 642 and/or other input controller(s) 644. Touch controller 642 can be coupled to a touch surface 646. Touch surface 646 and touch controller 642 may detect contact and movement or break thereof using any of a number of touch sensitivity technologies, including but not limited to capacitive, resistive, infrared, or surface acoustic wave technologies. Other proximity sensor arrays or elements for determining one or more points of contact with touch surface 646 may likewise be utilized. In one implementation, touch surface 646 can display virtual or soft buttons and a virtual keyboard, which can be used as an input/output device by the user.

Other input controllers 644 can be coupled to other input/control devices 648 such as one or more buttons, rocker switches, thumb-wheels, infrared ports, USB ports, and/or a pointer device such as a stylus. The one or more buttons (not shown) can include an up/down button for volume control of speaker 628 and/or microphone 630. In some implementations, device 600 can include the functionality of an audio and/or video playback or recording device and may include a pin connector for tethering to other devices.

Memory interface 602 can be coupled to memory 650. Memory 650 can include high-speed random access memory or non-volatile memory such as magnetic disk storage devices, optical storage devices, or flash memory. Memory 650 can store operating system 652, such as Darwin, RTXC, LINUX, UNIX, OS X, ANDROID, WINDOWS, or an embedded operating system such as VXWorks. Operating system 652 may include instructions for handling basic system services and for performing hardware dependent tasks. In some implementations, operating system 652 can include a kernel.

Memory 650 may also store communication instructions 654 to facilitate communicating with other mobile computing devices or servers. Communication instructions 654 can also be used to select an operational mode or communication medium for use by the device based on a geographic location, which could be obtained by the GPS/Navigation instructions 668. Memory 650 may include graphical user interface instructions 656 to facilitate graphic user interface processing such as the generation of an interface; sensor processing instructions 658 to facilitate sensor-related processing and functions; phone instructions 660 to facilitate phone-related processes and functions; electronic messaging instructions 662 to facilitate electronic-messaging related processes and functions; web browsing instructions 664 to facilitate web browsing-related processes and functions; media processing instructions 666 to facilitate media processing-related processes and functions; GPS/Navigation instructions 668 to facilitate GPS and navigation-related processes, camera instructions 670 to facilitate camera-related processes and functions; pedometer software 672 to process data received from a pedometer sensor; activation record or international mobile station equipment identity (IMEI) 674 for identifying the device 600 to other networked devices; and instructions for any other application (not shown) that may be operating on or in conjunction with the mobile computing device. Memory 650 may also store other software instructions for facilitating other processes, features and applications, such as applications related to navigation, social networking, location-based services or map displays.

Each of the above identified instructions and applications can correspond to a set of instructions for performing one or more functions described above. These instructions need not be implemented as separate software programs, procedures, or modules. Memory 650 can include additional or fewer instructions. Furthermore, various functions of the mobile device may be implemented in hardware and/or in software, including in one or more signal processing and/or application specific integrated circuits.

Certain features may be implemented in a computer system that includes a back-end component, such as a data server, that includes a middleware component, such as an application server or an Internet server, or that includes a front-end component, such as a client computer having a graphical user interface or an Internet browser, or any combination of the foregoing. The components of the system can be connected by any form or medium of digital data communication such as a communication network. Some examples of communication networks include LAN, WAN and the computers and networks forming the Internet. The computer system can include clients and servers. A client and server are generally remote from each other and typically interact through a network. The relationship of client and server arises by virtue of computer programs running on the respective computers and having a client-server relationship to each other.

One or more features or steps of the disclosed embodiments may be implemented using an API that can define on or more parameters that are passed between a calling application and other software code such as an operating system, library routine, function that provides a service, that provides data, or that performs an operation or a computation. The API can be implemented as one or more calls in program code that send or receive one or more parameters through a parameter list or other structure based on a call convention defined in an API specification document. A parameter can be a constant, a key, a data structure, an object, an object class, a variable, a data type, a pointer, an array, a list, or another call. API calls and parameters can be implemented in any programming language. The programming language can define the vocabulary and calling convention that a programmer may employ to access functions supporting the API. In some implementations, an API call can report to an application the capabilities of a device running the application, such as input capability, output capability, processing capability, power capability, and communications capability.

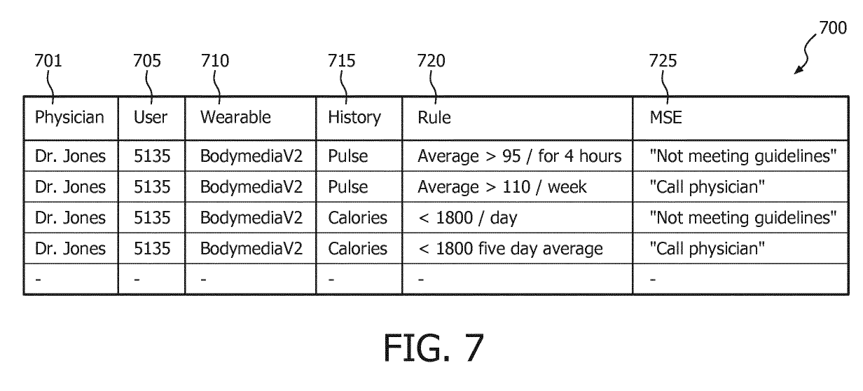

FIG. 7 illustrates an example history-action rule database 700 that may be used by embodiments described herein. The history-action database 700 is a variation of a “candidate rule database” or a “rule database” that may be used by one or more of the networks or devices in some embodiments. For example, the history-action database may describe the organization and contents of the action rule database 174 of the physician server 170 (as illustrated by history-action database 455 in FIG. 4A), candidate rule database 166 of the wearable device vendor server 180, rule database 166 of the user device 150, or rule database 136 of the wearable device 120. The history-action rule database of FIG. 7 identifies physicians 701, users 705, wearable devices 710, a history type 715, a rule 720, and a message action that is associated with the rule 725. A physician 701 identified in FIG. 7 and associated with each of the rules 720 is Dr. Jones. Similarly, a user 705 identified in FIG. 7 and associated with each of the rules 720 is user number 5135. Similarly, a wearable device identified in FIG. 7 and associated with each of the rules 720 is BodyMediaV2. In alternate embodiments, the history action rule database 700 may include data pertaining to multiple physicians 701, multiple users 705, and/or multiple wearable devices 710.

The history-action rule database of FIG. 7 also identifies two history types: pulse and calories. In other embodiments, other history types are possible, such as blood sugar, heart rhythm, or other health parameter history measurements.

The rules and corresponding action messages in FIG. 7 are the same rules and action messages discussed in respect to FIG. 4B: Rule 1 is triggered when the calories consumed by the user are <1800 calories per day. The action matched to this first rule is to send a message that the user is “not meeting guidelines.” Other rules that may be matched to actions in the figure are: Rule 2 is triggered when calories consumed are <1800 per day in a row of 5 days, which triggers performance the action of sending a message “call doctor's office,”; Rule 3 is triggered when an average pulse rate is >95 beats per minute for 4 hours, which triggers performance the action of sending the message “not meeting guidelines,”; Rule 4 is triggered when an average pulse rate is >110 beats per minute over a week, which triggers performance the action of sending the message “call doctor's office.” These entries should be understood to be illustrative rather than limiting.

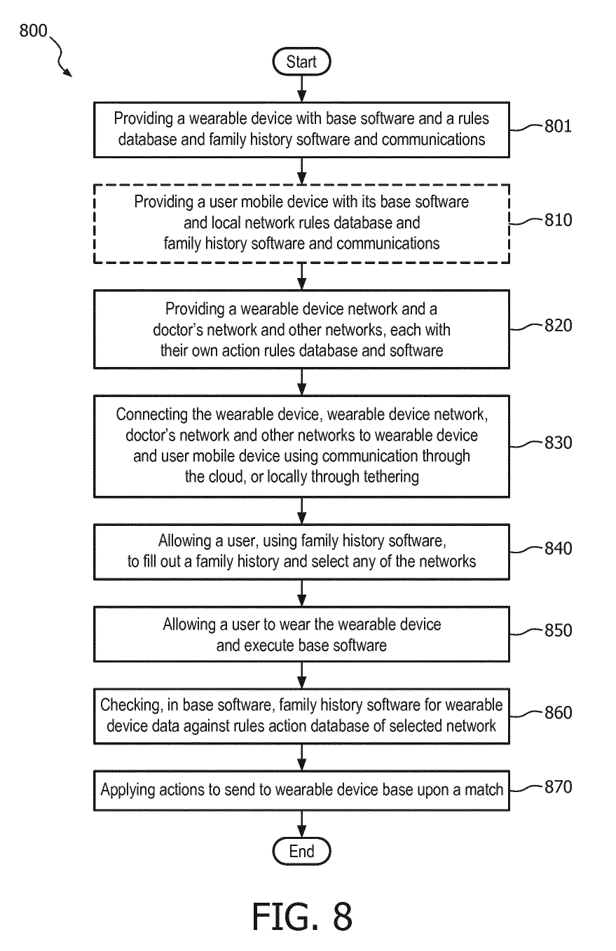

FIG. 8 illustrates an example method 800 of correlating family history and data collected by a wearable device to a health risk. After starting, step 801 in the method is where base software, a rules database, a family history software, and a communication interface may be provided to a wearable device. In optional step 810, a user mobile device may be provided with base software, a local network rules database, family history software, and a communication interface.

In step 820, a wearable device network, a doctor's network, and other networks, may each be provided with their own action rule database and software. In step 830, the wearable device may connect to the wearable device network, the doctor's network, and with other networks over the cloud using a communication interface. In this step, the wearable device and the user mobile device may communicate over the cloud may communicate locally through by being tethered using one or more communication interfaces.

In step 840, a user is allowed to fill out family history, select networks with which that family history may be shared while using a wearable device. In step 850, the user is allowed to wear the wearable device and to execute base software on the wearable device.

In step 860, wearable device data is checked against rules in the rules action database. Next, in step 870, actions matching the wearable device data and a rule are identified and cross referenced to an action, and the action is performed based on the match.

While the flow diagram 800 in FIG. 8 shows a particular order of operations performed by certain embodiments described herein, it should be understood that such order is an example (e.g., alternative embodiments can perform the operations in a different order, combine certain operations, overlap certain operations, etc.).

According to various embodiments, the family health data used by various embodiments may be retrieved from sources other than the user's indication of family history of health conditions. For example, in some embodiments, family history data may be retrieved from an electronic health record or from a wearable device record of the family member. In some such embodiments, such family members may be required to grant permission to the wearable device use approving such access before it is performed.

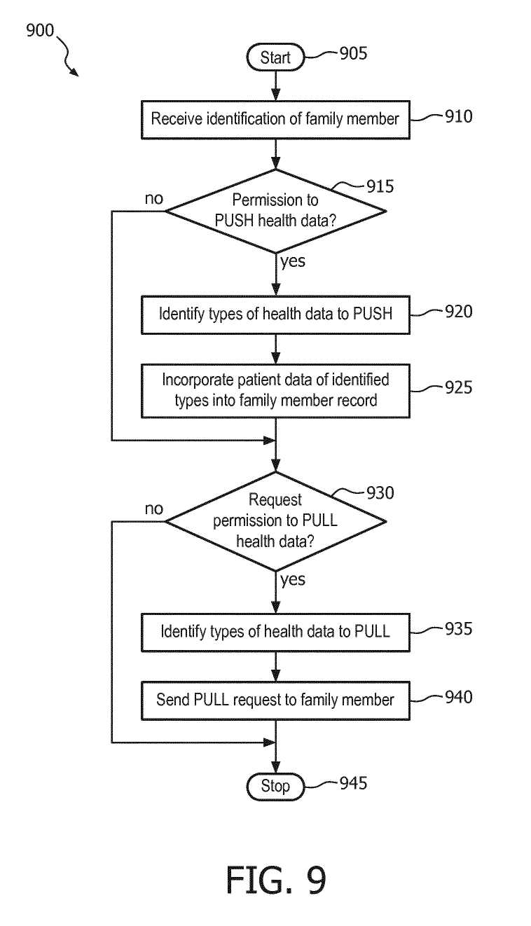

FIG. 9 is a flowchart illustrating an example of a method 900 for requesting and establishing sharing of health information between a patient and a family member. In various embodiments, the method 900 may correspond to the family history instructions 134 of the wearable device 120 or the family history instructions 164 of the user device 150 of FIG. 1. Alternatively, the method 900 may be performed by a web server or other server interacting with the user via a web portal, the wearable device 120, the user device 150, or other channels.

The method begins in step 905 and proceeds to step 910 where the device receives, from a user, and identification of a family member. For example, using a form, the user may input the names or other identifiers of one or more family member along with other information such as age, gender, relationship to the user, contact information, or indications of health conditions. Next, in step 915, the device determines whether the user has indicated a desire to grant permission to any of the entered family members to access any health information of the user. For example, via the aforementioned form, a use may indicate that electronic health records and wearable data should be shared with the user's father, that electronic health records only should be shared with the user's mother, and that no information should be shared with the user's cousin. Alternatively, in some embodiments, permission to share information with family members may be implied in whole or part through mere identification of the family member in step 910.

If the user has indicated a desire to share their own data with family members (i.e., “PUSH” data to those family members without their prior request), the method proceeds to step 920 where the device determines which types of health data (self-reported conditions, electronic health records, wearable heart monitor data, wearable pedometer data, etc.) should be shared and with whom. For example, step 920 may entail reading and interpreting the permissions indicated in to form of step 910. In some embodiments, step 920 may additionally include capturing the user's selection for auditing purposes (e.g., HIPAA compliance) so that, at all times, the system is able to determine which users gave permission to whom, for what, and when. In step 925, the device proceeds to incorporate patient data of the identified types into those identified family member records. For example, where the method 900 is performed by a wearable device or user device, the device may transmit a message to a server that stores or manages the family members' permissions to other patient's health data (e.g., the wearable device vendor server 180 for access to wearable device data or the physician server 170 for access to electronic health records). Where the method 900 is performed by a server managing the permissions, step 925 may include writing to an existing permissions record or creating a new permissions record for each identified family member indicating the level of access to the reporting user's health data. Thereafter, when the family member uses the systems described herein themselves, the user's health data will be available as family history data according to the various embodiments described herein.

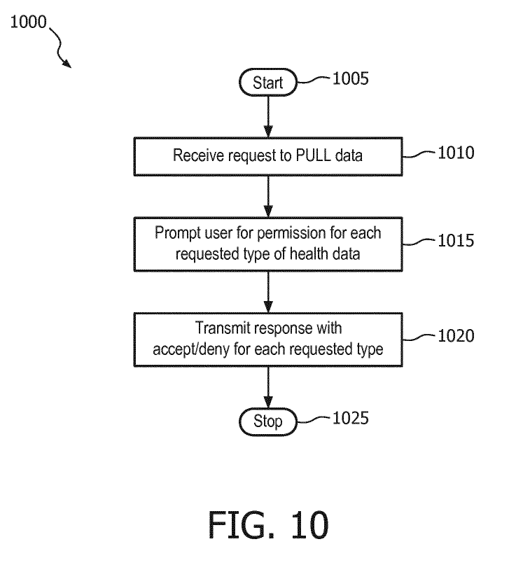

Next, in step 930, the device may determine whether the user has requested access to health data of the entered family members (requested, without a prior offer, to “PULL” the information for use). Like step 915, the request for permission to PULL information may be explicitly stated by the user, enumerated as a list of requested types of information, or simply implied by the user's entry of a family member. If there is at least one request to PULL information, the device determines, in step 935, which types of health data (self-reported conditions, electronic health records, wearable heart monitor data, wearable pedometer data, etc.) should be requested and from whom. Then, in step 940, the device sends the PULL request(s) to the family member(s). For example, where the method 900 is executed by a wearable device or user device, the device may send a message identifying the request details to one or more servers responsible for managing permissions to the family members' data. In some such embodiments, step 925, 940 may together construct a single message that includes both the PUSH permission and PULL request information. Where the method 900 is performed by a server managing the permissions, the device may transmit a notification via email, SMS text, a web portal, telephone call, or other medium to indicate to the family member that a permissions request has been received for them to approve or deny. In some such embodiments, step 940 may then lead to steps 1015, 1020 of FIG. 10, as will be described below. The method 900 then proceeds to end in step 945.

According to the foregoing, the method 900 accomplishes both sharing of the user's health data with family members and requesting that family members share their health data with the user. It will be apparent that various alternative embodiments may not attempt to accomplish both tasks, or may attempt to accomplish these tasks at separate times. For example, some embodiments may not implements the PUSH aspect and, instead, all family member health data must first be requested. Modifications to the method 900 to accomplish these alternative arrangements will be apparent.

FIG. 10 is a flow chart illustrating an example of a method 1000 for confirming or denying requests for access to health information. In various embodiments, the method 1000 may correspond to the family history instructions 134 of the wearable device 120 or the family history instructions 164 of the user device 150 of FIG. 1. Alternatively, the method 1000 may be performed by a web server or other server interacting with the user via a web portal, the wearable device 120, the user device 150, or other channels.

The method 1000 may begin in step 1005 and proceeds to step 1010 where the device receives a request to for permissions to PULL health data about a user. Such request may be received from, for example, a wearable device, a user device, or a server executing a version of the method 900; the request may be a result, for example, of step 940 of this method 900. In step 1015, the device prompts the user for an approve or deny response for each requested type of health data. For example, where the request includes a request for both health record and wearable data permissions, the user may be able to approve one and deny the other. The prompt of step 1015 may be accomplished through immediately communicating the request to the user via a user interface of a wearable device or user device, providing an alert that input is requested the next time the user visits an appropriate interface (e.g., a settings page of the wearable device or an app on the mobile device, or a landing page for a web-based portal), or through sending a message to another device (e.g., the server sending a message to the wearable device, the user device, or another server) to instruct that other device to obtain the user's approval or denial. Finally, in step 1020, the device transmits the user's response to the requesting device from which the request was received in step 1010. In some embodiments, step 1020 may additionally include capturing the user's selection for auditing purposes (e.g., HIPAA compliance) so that, at all times, the system is able to determine which users gave permission to whom, for what, and when. The method 1000 then proceeds to end in step 1025.

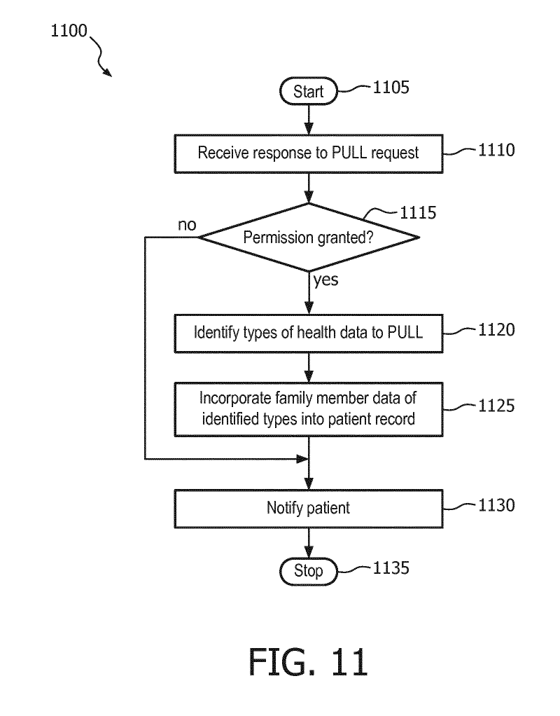

FIG. 11 is a flowchart illustrating an example of a method for establishing sharing of health information after grant of permission. In various embodiments, the method 1100 may correspond to the family history instructions 134 of the wearable device 120 or the family history instructions 164 of the user device 150 of FIG. 1. Alternatively, the method 1100 may be performed by a web server or other server interacting with the user via a web portal, the wearable device 120, the user device 150, or other channels.

The method 1100 begins in step 1105 and proceeds to step 1110 where the device receives a response to a previously submitted request for permissions to PULL health data. For example, such a response may be transmitted by step 1020 of method 1000. In step 1115, the device determines whether the response indicates that any of the requested permissions have been granted. If so, the method proceeds to step 1120 where the device determines to which types of health data (self-reported conditions, electronic health records, wearable heart monitor data, wearable pedometer data, etc.) the response grants the user access. For example, step 1120 may entail reading and interpreting permissions enumerated in the received response. In step 1125, the device proceeds to incorporate patient data from the approving family member of the identified types into the user's record. For example, where the method 1100 is performed by a wearable device or user device, the device may transmit a message to a server that stores or manages the user's permissions to other patient's health data (e.g., the wearable device vendor server 180 for access to wearable device data or the physician server 170 for access to electronic health records) indicating that the permissions have been granted. Where the method 1100 is performed by a server managing the permissions, step 1125 may include writing to an existing permissions record or creating a new permissions record for the user indicating the level of access to the responding family member's health data. Thereafter, when the user uses the systems described herein, the responding family member's health data will be available as family history data according to the various embodiments described herein. The device proceeds to notify the patient of the new permissions in step 1130 and the method 1100 proceeds to end in step 1135.

It will be apparent that in some embodiments or under some circumstances, a single server may be responsible for managing multiple users and the permissions associated therewith. For example, a user and a family member thereof may both have electronic health records managed by the same server. In such a context, the single server may not communicate with other servers to effect establishment of a new PULL permission. In such embodiments wherein the methods 900, 1000, 1100 are performed by such a server, the steps 940, 1010, 1020, or 1110 may be omitted and the respective methods 900, 1000, 1100 may be joined together into one process.

As explained above, various embodiments may utilize family history in a Boolean manner to determine whether certain rules should be installed on a wearable device or associated user device or whether certain installed rules are currently applicable and should be applied by the wearable device. For example, in the example of FIG. 3, two rules 335, 330 are created if there is a family history of both heart disease and high blood pressure. Various other embodiments, however, investigate the relevance of the family history further. For example, close biological relationship (e.g., parent-child) to a family member with a history of a medical condition may be a better indicator than a more distant relationship (e.g., second cousins) that a patient may experience a similar condition. As another example, if a family member's history of a medical condition may be attributable to behavioral factors (e.g., poor diet, tobacco usage, etc.) instead of genetic factors, the family history may not be as relevant to the patient. To account for such complexities, various embodiments may utilize a greater number of Boolean factors in a manner similar to that described with respect to FIG. 3 while other embodiments may utilize other approaches for determining the relevance of family history data such as, for example, calculating a numerical score to be compared to a threshold.

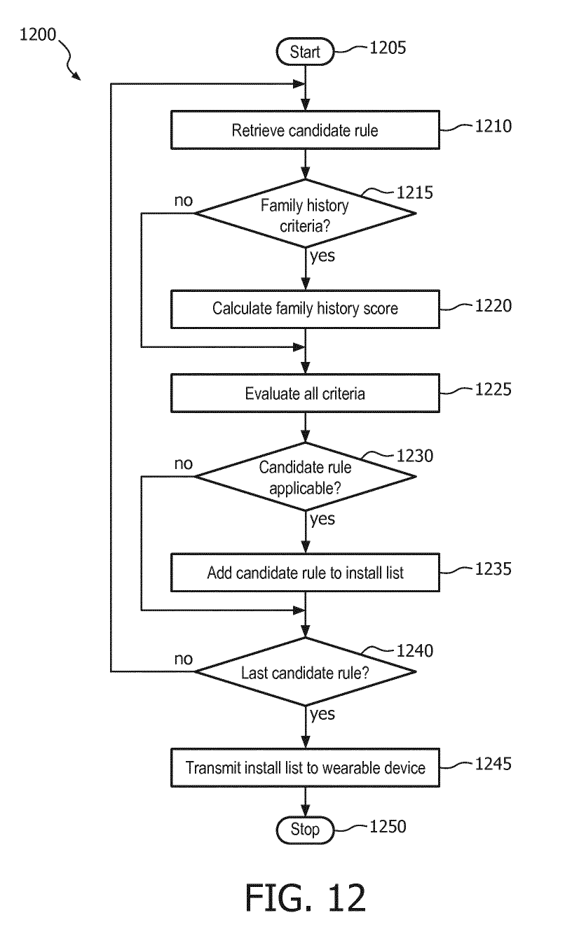

FIG. 12 is a flowchart illustrating an example of a method 1200 for identifying rules for installation using family history information. The method 1200 may correspond to, for example, the physician software 176, the WDN software 184, or third party software (not shown) for installing rules into the wearable device 120 or user device 150 of FIG. 1. Alternatively, the method 1200 may correspond to the family history instructions 164 of the user device 150 in embodiments where the user device selects rules to install on the wearable device 120. As yet another alternative, in some embodiments, the wearable device 120 may include rules that are activated (e.g., evaluated by the rules engine) and deactivated (e.g., skipped by the rules engine to conserve processing resources); such activation and deactivation may be accomplished, for example, by flagging each rule in the rules database 136, by maintaining a second database to store the active rules, or according to any other method. In such embodiments, the method 1200 may correspond to the family history instructions 134 and may be used to determine which locally-stored rules should be treated as active. As used herein, the term “installation” will be understood to encompass both activation of locally available rules and providing new rules to another device for future evaluation.

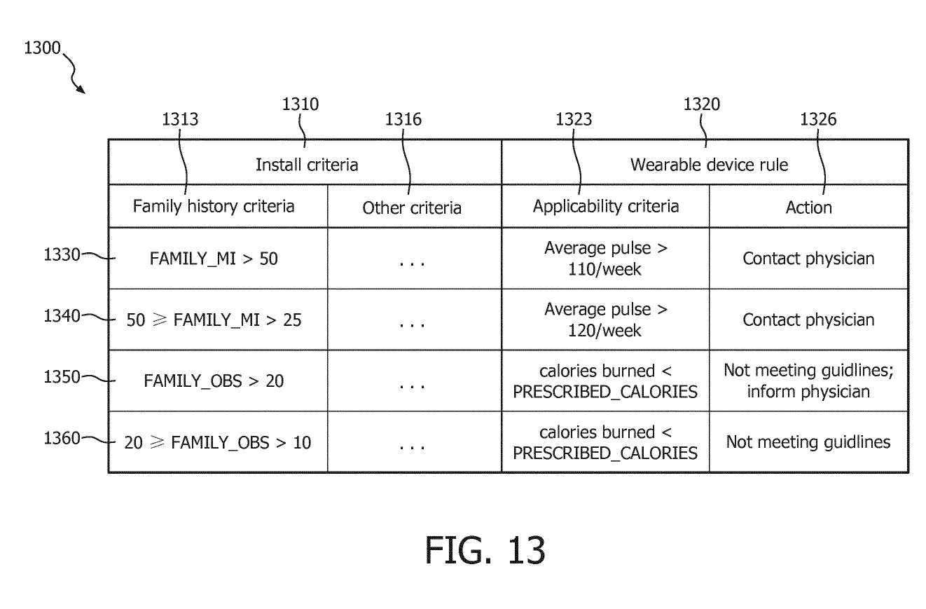

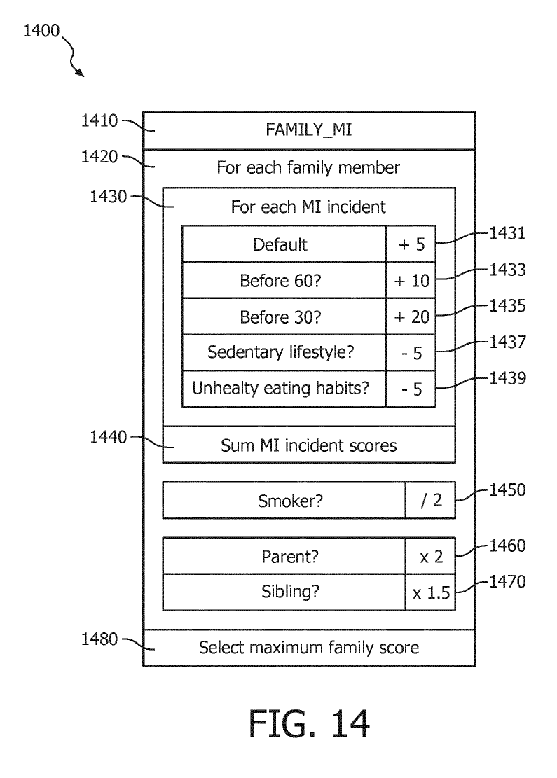

The method 1200 may begin in step 1205 in response to expiration of a periodic timer, a manual instruction, receipt of new family history or other context information regarding a user, receipt of new candidate rules in a candidate rule database, or in response to some other stimulus. The method 1205 proceeds to step 1210 where the device retrieves a first candidate rule to be evaluated for potential installation. As will be explained in greater detail below with respect to the example of FIG. 13, various candidate rules may include criteria for determining when installation of a behavioral rule is appropriate and should be effected on a wearable device. In step 1215, the device determines whether the installation criteria includes any criteria related to family history. If so, the device calculates one or more family history scores to be compared to the criteria in step 1220. For example, in various embodiments, formulae are provided for calculating family history scores related to heart health, obesity, diabetes, etc. An example of one embodiment of a formula for calculating family history risk associated with myocardial infarctions will be explained in greater detail below with respect to FIG. 14. After calculating the relevant family history scores in step 1220 (or determining that family history is irrelevant to installation of the present candidate rule in step 1215), the device evaluates all installation criteria in step 1225 and, based on the outcome of this step, determines whether the candidate rule is applicable for installation in step 1230.

When a candidate rule is applicable for installation, the device adds the candidate rule (or the wearable device rule contained therein or otherwise associated therewith), in step 1235, to a list of rules for installation in the wearable device for the present user. Next, in step 1240, the device determines whether additional candidate rules remain to be evaluated for installation. If so, the method 1200 loops back to step 1210 where the next candidate rule is retrieved for evaluation. After all candidate rules to be evaluated (e.g., all rules in the rules database, all new rules, etc.) have been processed, the method 1200 proceeds to step 1245 where the device transmits the install list to a wearable device for installation of the rules. The actual data transmitted may be the entire candidate rules, the wearable device rules associated with the candidate rules, identifiers of rules already sent to the wearable device, a location from which rules are to be downloaded, or any other data sufficient to instruct the wearable device to install the applicable rules. The method 1200 then proceeds to end in step 1250.

FIG. 13 illustrates an example of a candidate rule database 1300. In various embodiments, the candidate rule database 1300 may correspond to one of the candidate rule databases 174,182 of FIG. 1 or to another candidate rule database (not shown) stored at, for example, the third party server 190, the user device 150, or the wearable device 120. While the database 1300 is shown in a tabular format, it will be appreciated that virtually any appropriate data structure may be used to represent a candidate rule database 1300. For example, in some embodiments, the install criteria 1310 may be stored in a first table while the wearable device rules 1320 may be stored in a second table. Such an arrangement may be used when a single set of install criteria is used to determine whether a group of wearable device rules should be installed; for example, each set of install criteria in the first table may be associated with one or more identifiers that, in the second table, are associated with wearable device rules or groupings thereof. Thus, a candidate rule may identify a wearable device rule either by storing the entire rule therein or by referencing another location where the rule may be found.

As shown, each example rule includes two sections: install criteria fields 1310 for determining whether rules should be installed and wearable device rule fields 1320 for defining or otherwise identifying one or more rules to be installed on a wearable device. The install criteria fields 1310 include a family history criteria field 1313 and other criteria field 1316. It will be understood that in various embodiments, family history criteria may not be separated into its own field 1313 and, instead, all installation criteria may be stored together in a single expression.

The family criteria field 1313 stores one or more conditions to be evaluated against family history data. For example, in some embodiments, the family criteria field 1313 may store a condition evaluating whether one or more flags have been set by the wearable device user (e.g., via the interface 205 of FIG. 2). In some embodiments, the family criteria field 1313 may additionally or alternatively store a condition including a more complex formula or reference thereto to be evaluated (e.g., in step 1220 of FIG. 12). The other criteria field 1316 may store various additional criteria to be evaluated when determining whether a candidate rule is applicable such as, for example, conditions that evaluate immediate or historical data from the wearable device (or from another wearable device such as, for example, historical data downloaded from a wearable device previously worn by the user), the wearable device user's medical history (e.g., electronic health records), input from the wearable device user's physician (e.g., previously set Boolean flags), or virtually any other condition appropriate for judging the applicability of a candidate rule.

The wearable device rule fields 1320 may include one or more fields useful to define or otherwise identify a wearable device rule to be installed when a candidate rule is applicable. For example, in various embodiments, the wearable device rule fields 1320 may include only a single identifier field that stores a rule ID that corresponds to a rule definition in another database or otherwise in another location. In the illustrated examples, the wearable device rule fields 1320 include an applicability criteria field 1323 for determining, after installation of the wearable device rule, whether the wearable device rule is applicable and an action rule 1326 for defining an action to be taken when the wearable device rule is applicable. Thus, the illustrated examples include two different types of criteria: installation criteria for determining when the wearable device rule should be installed (e.g., at a wearable device or user device) and applicability criteria for determining, after such installation, whether an action should be performed.

As a first example, a first candidate rule 1330 indicates that a wearable device rule should be installed when a FAMILY_MI score exceeds 50. As will be explained in greater detail below, the string “FAMILY_MI” may refer to a family myocardial infarction risk score calculated according to a formula which may be defined in the field or elsewhere and referenced by the name. When this score exceeds 50, then a wearable device rule that contacts the user's physician when the user's average weekly pulse exceeds 110 will be installed on the user's wearable device or user device. It will be appreciated that the use of scores may enable an enhanced flexibility and tailoring of rules to the specific family history reported, beyond simple Boolean indications of whether a family history exists. For example, example candidate rule 1340 also evaluates the FAMILY_MI score, but in contrast to the first example 1330, determines whether the score falls between 25 and 50. When this candidate rule is applicable, the installed wearable device rule will contact the physician when the average weekly pulse exceeds 120, instead of 110.

A similar set of examples 1350, 1360 uses a different family history score, FAMILY_OBS, which may calculate an obesity risk score associated with family members of the user. In this example, different thresholds are used to set different actions: in the candidate rule 1350 where the FAMILY_OBS score exceeds 20, the installed rule will contact the user's physician when the measured calories burned does not exceed a calorie amount prescribed by the doctor (thereby cross referencing other user data such as, for example, the user's electronic health record). The candidate rule 1360, however, will result in a rule that only informs the user that they are not meeting guidelines when this same applicability criteria.

FIG. 14 illustrates an example of a family history criteria formula 1400. It will be apparent that FIG. 14 is an abstraction and that the formula may be stored according to various data structures such as, for example, a text string, a formula object, code or pseudo code, or virtually any other structure that can be evaluated to produce an output.

In the example shown, the formula 1400 includes an identifier 1410 specifying that the formula is to be applied when criteria references the string “FAMILY_MI.” The formula includes a loop 1420 that calculates a score associated with each known family member for the user. For each family member, the formula evaluates each myocardial infarction (“MI”) event 1430 (e.g., as recorded in an electronic health record). For each such incident, a default score of 5 1431 is assumed. In the next two steps 1433, 1435, values of 10 and 20 are added to the default score if the event occurred before the family member reached the ages of 60 and 30 respectively. Next, the formula takes factors that may tend to suggest that the risk is not hereditary into account by, in steps 1437, 1439, reducing the score by 5 if the family member held a sedentary lifestyle or followed unhealthy eating habits at the time of the event. Next, in step 1440, the values of all MI events are summed into an aggregate score.

In step 1450, the aggregate score is halved if the family member is a smoker. Next, in steps 1460, 1470 the relationship proximity is taken into account. The score is doubled if the family member is a parent or multiplied by 1.5 if the family member is a sibling. Finally, in step 1480, the single maximum score from any family member is selected as the overall risk score to be returned and used in evaluating criteria.

It will be apparent that the formula 1400 is just one example of a formula. In various embodiments, alternative formulae may be used. Such formulae may be manually defined by physicians or other experts or may be computer generated using various machine-learning approaches such as, for example, neural networks, deep learning, Bayesian networks, etc.

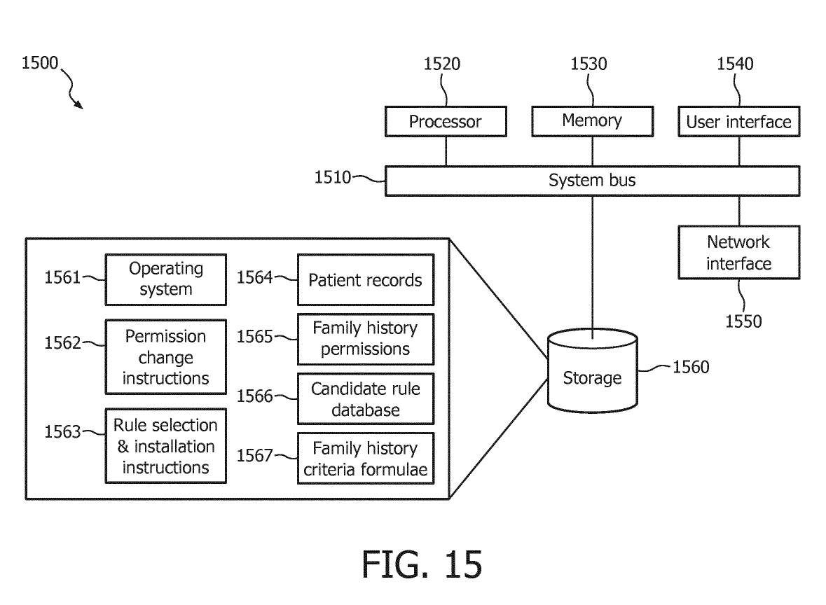

FIG. 15 illustrates an example of hardware for implementing a rule installation server 1500. As used herein, the term “rule installation server” will be understood to refer to any device that selects wearable device rules for installation on a wearable device. In various embodiments, the physician server 170, wearable device vendor server 180, third party server 190, user device 150, or wearable device 120 (e.g., in the embodiment where the wearable device selects rules to activate) may constitute rule installation servers. As shown, the device 1500 includes a processor 1520, memory 1530, user interface 1540, network interface 1550, and storage 1560 interconnected via one or more system buses 1510. It will be understood that FIG. 2 constitutes, in some respects, an abstraction and that the actual organization of the components of the device 1500 may be more complex than illustrated.

The processor 1520 may be any hardware device capable of executing instructions stored in memory 1530 or storage 1560 or otherwise processing data. As such, the processor may include a microprocessor, field programmable gate array (FPGA), application-specific integrated circuit (ASIC), or other similar devices.

The memory 1530 may include various memories such as, for example L1, L2, or L3 cache or system memory. As such, the memory 1530 may include static random access memory (SRAM), dynamic RAM (DRAM), flash memory, read only memory (ROM), or other similar memory devices.

The user interface 1540 may include one or more devices for enabling communication with a user such as an administrator. For example, the user interface 1540 may include a display, a mouse, and a keyboard for receiving user commands. In some embodiments, the user interface 1540 may include a command line interface or graphical user interface that may be presented to a remote terminal via the network interface 1550.

The network interface 1550 may include one or more devices for enabling communication with other hardware devices. For example, the network interface 1550 may include a network interface card (NIC) configured to communicate according to the Ethernet protocol. Additionally, the network interface 1550 may implement a TCP/IP stack for communication according to the TCP/IP protocols. Various alternative or additional hardware or configurations for the network interface 1550 will be apparent.

The storage 1560 may include one or more machine-readable storage media such as read-only memory (ROM), random-access memory (RAM), magnetic disk storage media, optical storage media, flash-memory devices, or similar storage media. In various embodiments, the storage 1560 may store instructions for execution by the processor 1520 or data upon with the processor 1520 may operate. For example, the storage 1560 may store a base operating system 1561 for controlling various basic operations of the hardware 1500. Permission change instructions 1562 may include instructions for requesting, granting, and recording various users permissions to access other users' and family members' health data. For example, the permission change instructions 1562, in various embodiments, may incorporate one or more of methods 900, 1000, 1100. The rule selection and installation instructions 1563 may include instructions for determining which wearable device rules are to be installed and effecting such installation. For example, the rule selection and installation instructions 1563 may correspond to the method 1200. The storage may also include various data to support the operations of the instructions 1561, 1562, 1563 such as, for example, patient records 1564, family history permissions 1565, a candidate rule database 1566, and family history criteria formulae 1567. It will be apparent that, in various embodiments, some or all of this data 1564-67 may instead me hosted by other devices and accessible to the server 1500 via the network interface 1550 or another interface.

It will be apparent that various information described as stored in the storage 1560 may be additionally or alternatively stored in the memory 1530. In this respect, the memory 1530 may also be considered to constitute a “storage device” and the storage 1560 may be considered a “memory.” Various other arrangements will be apparent. Further, the memory 1530 and storage 1560 may both be considered to be “non-transitory machine-readable media.” As used herein, the term “non-transitory” will be understood to exclude transitory signals but to include all forms of storage, including both volatile and non-volatile memories.

While the host device 1500 is shown as including one of each described component, the various components may be duplicated in various embodiments. For example, the processor 1520 may include multiple microprocessors that are configured to independently execute the methods described herein or are configured to perform steps or subroutines of the methods described herein such that the multiple processors cooperate to achieve the functionality described herein. Further, where the device 1500 is implemented in a cloud computing system, the various hardware components may belong to separate physical systems. For example, the processor 1520 may include a first processor in a first server and a second processor in a second server.

It should be apparent from the foregoing description that various exemplary embodiments of the invention may be implemented in hardware and/or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method of configuring wearable device behavior based on family history, the method comprising:
receiving, at a rule installation server, family health data associated with at least one family member of a wearable device user;
retrieving a candidate rule comprising installation criteria and an identification of a wearable device rule;
evaluating the installation criteria using the family health data to determine that the candidate rule is to be installed; and
based on determining that the candidate rule is to be installed, communicating the wearable device rule for installation on the wearable device.

2. The method of claim 1, wherein communicating the wearable device rule for installation comprises transmitting a message to the wearable device to effect installation of the wearable device rule.

3. The method of claim 1, wherein receiving the family health data comprises receiving, from a user of the wearable device, an identification of one or more health conditions experienced by a family member.

4. The method of claim 1, wherein receiving the family health data comprises accessing an electronic health record of at least one family member.

5. The method of claim 1, further comprising:
receiving, from the at least one family member, an identification of relationship to the wearable device user; and
based on receiving the identification of relationship to the wearable device user, modifying a user record of the wearable device record to reflect a permission to access health data of the at least one family member,
wherein receiving the health data comprises retrieving the health data of the at least one family member based on the permission.

6. The method of claim 1, further comprising:
receiving, from the wearable device user, an identification of relationship to the at least one family member;
transmitting, to the at least one family member, a request to grant permission to access health data of the at least one family member;
receiving, from the at least one family member, an approval to access health data of the at least one family member;
based on receiving the approval, modifying a user record of the wearable device record to reflect a permission to access health data of the at least one family member,
wherein receiving the health data comprises retrieving the health data of the at least one family member based on the permission.

7. The method of claim 1, wherein evaluating the installation criteria using the family health data comprises evaluating at least one modifiable risk factor of the at least one relative.

8. A rule installation server comprising:
a memory storing a candidate rule comprising installation criteria and an identification of a wearable device rule;
a network interface; and
a processor configured to:
receive family health data associated with at least one family member of a wearable device user,
evaluate the installation criteria using the family health data to determine whether the candidate rule is to be installed;
based on a determination that the candidate rule is to be installed, communicating the wearable device rule for installation on the wearable device.

9. The rule installation server of claim 8, wherein, in communicating the wearable device rule for installation, the processor is configured to transmit a message to the wearable device to effect installation of the wearable device rule.

10. The rule installation server of claim 8, wherein, in receiving the family health data, the processor is configured to receive, from a user of the wearable device, an identification of one or more health conditions experienced by a family member.

11. The rule installation server of claim 8, wherein, in receiving the family health data, the processor is configured to access an electronic health record of at least one family member.

12. The rule installation server of claim 8, wherein the processor is further configured to:
receive, from the at least one family member, an identification of relationship to the wearable device user; and
based on receiving the identification of relationship to the wearable device user, modify a user record of the wearable device record to reflect a permission to access health data of the at least one family member,
wherein, in receiving the health data, the processor is configured to retrieve the health data of the at least one family member based on the permission.

13. The rule installation server of claim 8, wherein the processor is further configured to:
receive, from the wearable device user, an identification of relationship to the at least one family member;
transmit, to the at least one family member, a request to grant permission to access health data of the at least one family member;
receive, from the at least one family member, an approval to access health data of the at least one family member;
based on receiving the approval, modify a user record of the wearable device record to reflect a permission to access health data of the at least one family member,
wherein, in receiving the health data, the processor is configured to retrieve the health data of the at least one family member based on the permission.

14. The rule installation server of claim 8, wherein, in evaluating the installation criteria using the family health data, the processor is configured to evaluate at least one modifiable risk factor of the at least one relative.

15. A non-transitory machine-readable medium encoded with instructions for execution by a rule installation server, the non-transitory machine-readable medium comprising:
instructions for receiving, at a rule installation server, family health data associated with at least one family member of a wearable device user;
instructions for retrieving a candidate rule comprising installation criteria and an identification of a wearable device rule;
instructions for evaluating the installation criteria using the family health data to determine whether the candidate rule is to be installed; and
instructions for, based on a determination that the candidate rule is to be installed, communicating the wearable device rule for installation on the wearable device.
